(19) United States
(12) Patent Application Publication
(10) Pub. No.: US 2025/0368931 A1
(43) Pub. Date: Dec. 4, 2025

(54) ENVIRONMENTALLY SUSTAINABLE SYSTEMS AND METHODS OF BIOGAS PRODUCTION AND BUFFERED UTILIZATION

(71) Applicant: HOME BIOGAS LTD, Beit Yanai (IL)

(72) Inventors: Oshik Moshe EFRATI, BET YANAI (IL); Yossi ROSENBLUM, Pardesiya (IL); Ilan PORTIANSKY, Ramat Ishay (IL); Guy NEUMANN, Lehavot Haviva (IL); Shoham ZAK, GIVAT ELA (IL); Bar GERSHON, Kfar Saba (IL); Eran ISRAELI, Kfar Vitkin (IL); Eyal GERZI, Bet Hashita (IL)

(73) Assignee: HOME BIOGAS LTD, BEIT YANI (IL)

(21) Appl. No.: 18/675,221

(22) Filed: May 28, 2024

(51) Int. Cl. C12M 1/107; B09B 3/35; B09B 3/65; C02F 11/04; C12M 1/00; C12M 1/34

(52) U.S. Cl. CPC C12M 21/04; B09B 3/35; B09B 3/65; C02F 11/04; C12M 41/34; C12M 43/00; C02F 2303/26

(57) ABSTRACT

An environmentally sustainable biogas production and buffered utilization system is described; a respective environmentally sustainable method of biogas production and buffered utilization is further described; the system comprises: an anaerobic digestor, a feeder sub-assembly operationally connected to the inlet, a utilization module operationally connected to the gas outlet, an intermittent gas accumulation module and a controller; the method comprises: providing a biogas production and buffered utilization system, filling the anaerobic digestor with liquids, buffering a produced biogas in the intermittent gas accumulation module, detecting by the sensor the variable volume gas reservoir in the erected configuration and controllably igniting the gas consumer element by the igniter.

### TECHNICAL FIELD

In general, the present invention pertains to systems and methods of recycling organic waste and utilizing the products thereof. In particular, the invention relates to environmentally sustainable systems and methods of biogas production and buffered utilization.

### BACKGROUND OF THE INVENTION

Organic waste makes up a considerable percentage of total waste. This waste is typically thrown out with the rest of the garbage, requiring transport and space in dumps. Such waste is occasionally used for the purposes of producing compost, saving the transport and space requirements, as well as providing a source of rich soil. Hence improved system and methods for combined biogas and fertilizer production from such waste organic waste shall entail an environmental benefit.

CN201344363Y teaches a biogas storage tank comprising a telescopic tank, an air duct and an air valve. The air duct in CN201344363Y is communicated with the telescopic tank, and the air valve is mounted on the air tube. The biogas storage tank in CN201344363Y also comprises an elastic rope, one end of which is connected with the bottom of the telescopic tank. The biogas storage tank in CN201344363Y further comprises a pressure gauge which is arranged on the telescopic tank. In CN201344363Y the surplus biogas in a biogas generation tank can be stored in the biogas storage tank, thereby realizing environmental-protection, energy conservation and convenient use; and the biogas stored in the biogas storage tank can be sold as commodity.

U.S. Pat. No. 20,230,071928 teaches system of controllable separation between recyclable organic waste and graywater sewage is described; a respective method for controllable separating recyclable organic waste from graywater sewage is further described; the system includes: a garbage disposal unit, and a separation module; the method includes: draining both the graywater sewage and the recyclable organic waste; processing the organic waste into a semiliquid mixture or slurry of round organic matter and fluid; discharging the semiliquid mixture or slurry of ground organic matter and fluid and the graywater sewage; and releasing the semiliquid mixture or slurry of ground organic matter and fluid; releasing the graywater sewage; and separating the semiliquid mixture or slurry of ground organic matter and fluid from the graywater sewage.

### SUMMARY OF THE INVENTION

The following summary of the invention is provided to exhibit the basic understanding of some principles, underlying various aspects and features of the invention. This summary is not an extensive overview of the invention and as such it is not necessarily intended to particularly identify all key or critical elements of the invention and is not to delineate the scope of the invention. Its sole purpose is to present some concepts of the invention in a simplified form as a prelude to the following more detailed.

The invention was made in view of the deficiencies of the prior art and provides systems, methods and processes for overcoming these deficiencies. According to some embodiments and aspects of the present invention, there is provided an environmentally sustainable biogas production and buffered utilization system including: an anaerobic digestor including: an essentially cylindrically shaped firm encasement, an inlet disposed at a bottom portion of the encasement, a gas outlet disposed at a top portion of the encasement, configured to duct a biogas, produced by essentially anaerobic digest processes in the digestor, an overflow outlet of the encasement, configured to drain surplus liquids from the digestor, where the anaerobic digestor is configured to be essentially filled with liquids, without a substantial space for a discrete gaseous fraction in the encasement; a feeder sub-assembly operationally connected to the inlet, including: a sink configured to receive organic waste, a grinder, operationally connected to the sink and to the inlet, configured to receive the organic waste from sink, to grind the organic waste and to feed ground organic waste into the inlet; a utilization module operationally connected to the gas outlet, configured to receive the biogas from the gas outlet and to burn it upon demand, including: a controllable igniter, a gas consumer element, actuatable by the controllable igniter, a valve configured to control an inflow of the biogas to the gas consumer element; an intermittent gas accumulation module including: a variable volume gas reservoir, including an elongated pliant accordion shaped container, the variable volume gas reservoir is configured to assume an erected configuration, in which the variable volume gas reservoir is essentially filled with the biogas, and a collapsed configuration, in which variable volume gas reservoir is essentially depleted of the biogas, at least one gas channel, operationally connected to the gas outlet and to the utilization module, a weight disposed on top of the variable volume gas reservoir, configured to exert a predetermined gravitational force onto the variable volume gas reservoir, thereby forming a substantially constant pressure, whilst the variable volume gas reservoir is in-between the erected and collapsed configurations, at least one sensor, disposed on top of the weight, configured to detect the variable volume gas reservoir in the erected configuration; a controller, operationally connected at least to the sensor of the intermittent gas accumulation module and the controllable igniter of the utilization module.

In some embodiments, the system further includes a sewage sub-system, configured to receive at least one substance selected from the group consisting of: greywater, blackwater and wastewater.

In some embodiments, the anaerobic digestor further includes an interior heater.

In some embodiments, the utilization module includes at least one gas consumer device selected from the group consisting of: a gas burner, gas water heater, gas turbine and gas powered electrical generator.

In some embodiments, the variable volume gas reservoir includes a composite material, including a pliant polymeric sheet and a metallic gas barrier film.

In some embodiments, the controller is further operationally connected to the valve of the utilization module.

In some embodiments, the intermittent gas accumulation module further includes a mechanical restrictor, configured to confine the variable volume gas reservoir, thereby essentially preventing buckling and/or sidewise deformation of the variable volume gas reservoir, from a substantially linearly straight vertically oriented conformation.

In some embodiments, the mechanical restrictor includes a vertically slidable bracket, movable with the variable volume gas reservoir, whilst the variable volume gas reservoir is in-between the erected and collapsed configurations.

In some embodiments, the mechanical restrictor is disposed at a distance of approximately ⅔ of a maximal length of the variable volume gas reservoir, from a bottom of the variable volume gas reservoir, whilst the variable volume gas reservoir is in the erected configuration.

In some embodiments, the overflow outlet is disposed at a centrical portion along a length of the encasement, configured to avoid capturing solids whilst draining the surplus liquids from the digestor.

According to some embodiments and aspects of the present invention, there is provided an environmentally sustainable method of biogas production and buffered utilization including: providing a biogas production and buffered utilization system including: an anaerobic digestor, a feeder sub-assembly operationally connected to the inlet of the anaerobic digestor, a utilization module operationally connected to the gas outlet of the anaerobic digestor, configured to receive the biogas from the gas outlet and to burn it upon demand, an intermittent gas accumulation module, a controller, operationally connected at least to a sensor of the intermittent gas accumulation module and a controllable igniter of the utilization module; filling the anaerobic digestor with liquids, without leaving a substantial discrete space for a gaseous fraction in the encasement; buffering a produced biogas by the intermittent gas accumulation module, by changing a volume of the variable volume gas reservoir; detecting by the sensor the variable volume gas reservoir in the erected configuration; controllably igniting the gas consumer element by the igniter.

In some embodiments, the method further includes interiorly heating the anaerobic digestor.

In some embodiments, where the controller is further operationally connected to the valve of the utilization module, the method further includes: opening the valve of the utilization module and forming the inflow of the biogas to the gas consumer element, and closing the valve of the utilization module and obstructing the inflow of the biogas to the gas consumer element.

In some embodiments, where the intermittent gas accumulation module further includes a mechanical restrictor, configured to confine the variable volume gas reservoir, the method further includes preventing buckling and/or sidewise deformation of the variable volume gas reservoir, from a substantially linearly straight vertically oriented conformation, by the mechanical restrictor.

In some embodiments, where the mechanical restrictor includes a vertically slidable bracket, the method further includes moving the vertically slidable bracket with the variable volume gas reservoir, whilst the variable volume gas reservoir is in-between the erected and collapsed configurations.

In some embodiments, the method further includes disposing the mechanical restrictor at a distance including approximately of ⅔ of a maximal length of the variable volume gas reservoir, from a bottom of the variable volume gas reservoir, whilst the variable volume gas reservoir is in the erected configuration.

In some embodiments, the method further avoiding capturing solids whilst draining the surplus liquids from the digestor.

In some embodiments, the mechanical restrictor comprises a plurality of vertically slidable brackets, disposed equidistantly alongside the variable volume gas reservoir in the erected configuration.

The term matching or a term similar thereto, as referred to herein, is to be construed as having a cross-sectional area and/or shape of a component equal or essentially similar to a cross-sectional area and/or shape of another component. It should be acknowledged that the components may only to be similar in the cross-sectional areas and/or shapes, to satisfy the term matching or similar, so long as the cross-sectional areas of the components can be mated and/or inserted into each other and/or the combination thereof essentially fits together and/or occupy essentially the same space.

The term structured, as referred to herein, is to be construed as including any geometrical shape, exceeding in complexity a plain linear shape or a shape embodying a simple and/or standardized circular, elliptical or polygonal contour or profile. Any more complex shape than a plain linear shape or a shape embodying a simple and/or standardized circular, elliptical or polygonal contour or profile, constitutes an example of structured geometry.

The term modular, as referred to herein, should be construed as a including a stand-alone and/or autonomically functioning of structured unit. The term modular inter alia means a standardized unit that may be conveniently installed or deployed without significant impact to the environment. The term modular, however, doesn't necessarily mean providing for ease of interchange or replacement. The term modular is optionally satisfied solely by providing for ease of onetime deployment or installation.

The terms connected, coupled, connectable and/or “in connection with” refer to any form of interaction between two or more entities, including mechanical, electrical, magnetic, electromagnetic, fluid, and thermal interactions. Components can be operatively coupled to each other even though they are not in direct contact with each other. The term “abutting” refers to items that are in direct physical contact with one another, although the items do not necessarily have to be attached to one another.

By operationally connected and operably coupled or similar terms used herein is meant connected in a specific way (e.g., in a manner allowing fluid to move and/or electric power or signal to be transmitted) that allows the disclosed system and its various components to operate effectively in the manner described herein.

The term readily connectable, as referred to herein, should be construed as including any structure and/or member that is configured to be conveniently connected to other structure and/or member and/or components of a larger system or assembly. The term readily connectable, however, doesn't necessarily mean readily disconnectable or removable. The term readily connectable is optionally satisfied by providing for ease of onetime connection or coupling.

The term fastener or a term similar thereto, as referred to herein, is to be construed as any suitable structure, material and/or device that effects an attachment, mounting and/or affixing, in a non-limiting manner including the examples of: bolts, screws, staples, pins, clips, magnetic couplings, zippers, snaps, magnets, non-permanent adhesives, adhesives, welding, nails, rivets, buckles, straps, stings, knots, hook and loop fasteners such as VELCRO (RTM), which is a trademark registered to Velcro Industries B.V.

The term biasing means or alike, as referred to herein, should be construed as including any material, structure or mechanism, configured to accumulate mechanical energy, by changing the configuration thereof, upon a force exerted thereon, such as a compressive, tensile, shear or torsional force, as well as for releasing the energy accumulated therein, by returning to the normal or default configuration thereof and thereby performing a mechanical work, typically by linear or radial displacement. Examples of biasing means in a non-limiting manner include, springs, elastomers, leaf-springs, coil-springs, tension/extension spring, compression spring torsion spring, constant spring, variable spring, variable stiffness spring, flat spring, machined spring, serpentine spring, garter spring, cantilever spring, helical spring, hollow tubing springs, volute spring, V-spring, belleville washer or belleville spring, constant-force spring, gas spring, mainspring, negator spring, progressive rate coil springs, rubber band, spring washer and wave spring.

The term environmentally sustainable, as referred to herein, is to be construed as including any material that is biodegradable or comprising a naturally occurring and/or excavated and/or mined material, whether in original, natural or processed form. The term environmentally sustainable, as referred to herein, is to be equally construed as including in a non-limiting manner any method or technique facilitating a reduction in: (1) energy consumption including energy consumption, whether required for manufacture, storage and/or transportation, (2) the volume or mass of disposed materials, waste or emissions, as well as (3) toxicity or non-biodegradability of disposed materials, waste or emissions.

The term fluid or liquid, as referred to herein, is to be construed as any material that deforms when a shear stress is applied. While fluid generally would refer to any liquids or gases, it may be used herein to describe fluidized solids and bulk solids and/or granulate matter that are capable of flowing or otherwise moving inside a device as a result of pressure differences and/or gravitational force. Such materials may include slurries, suspensions, pastes, powders, granular solids, particle solids, granulate matter, particulate matter, as well as any combinations thereof.

The terms firm rigid, or stiff, as referred to herein, are to be construed as having rigidity modulus value, otherwise referred to as the shear modulus, of 4800 MPa or more. Materials are considered to be firm rigid, or stiff but not tensile, when such materials are incapable of being efficiently elastically flexed or bent. Stiff materials, such as steel, are defined as having rigidity modulus value well exceeding 4800 MPa.

The terms pliable or pliant, as referred to herein, are to be construed as having high tensile strength and capable of being efficiently elastically flexed or bent but not being resilient and incapable of being efficiently stretched or expanded. The term tensile or tensile strength, as referred to herein, is to be construed inter alia as a shortcut of the known term ultimate tensile strength, frequently represented acronym as UTS, meaning an intensive property of a material or structure to withstand loads tending to elongate, namely to resist tension, defined as the maximum stress that a material can withstand while been stretched or pulled before sustaining breaking, substantial deformation and/or necking before fracture, such as nylon, relating to essentially non-ductile materials, having UTS value ranging between about 600 and 1000 MPa or more, but not including rigid, firm or stiff materials.

The terms elastic or resilient, as referred to herein, are to be construed as having tensile strength lower than aforesaid tensile strength of pliable or pliant material and optionally being capable of efficiently stretching or expanding, relating inter alia to essentially ductile materials, having UTS value lesser than about 600 MPa.

The term water shall particularly include water that is fit for consumption by a living organism and/or make the water potable. In certain embodiments the living organism is a “mammal” or “mammalian”, where these terms are used broadly to describe organisms which are within the class mammalia, including the orders carnivore, rodentia and primates or humans. In some embodiments of the disclosed systems, desalination is removing an amount of salt and/or other minerals or components from saline water so that the water is fit for a specific purpose (e.g., irrigation or industry).

The term slurry, as referred to herein, is to be construed as a mixture of solids denser than water suspended in liquid, usually water. Solids concentrations in a slurry typically range between about 0.5 percent and about 5 percent.

The term sludge, as referred to herein, is to be construed as a semi-solid slurry. The term is also sometimes used as a generic term denoting solids separated from suspension in a liquid. Solids concentrations in a sludge typically range between about 5 percent and about 15 percent.

The terms method and process as used herein are to be construed as including any sequence of steps or constituent actions, regardless a specific timeline for the performance thereof. The particular steps or constituent actions of any given method or process are not necessarily in the order they are presented in the claims, description or flowcharts in the drawings, unless the context clearly dictates otherwise. Any particular step or constituent action included in a given method or process may precede or follow any other particular step or constituent action in such method or process, unless the context clearly dictates otherwise. Any particular step or constituent action and/or a combination thereof in any method or process may be performed iteratively, before or after any other particular step or action in such method or process, unless the context clearly dictates otherwise. Moreover, some steps or constituent actions and/or a combination thereof may be combined, performed together, performed concomitantly and/or simultaneously and/or in parallel, unless the context clearly dictates otherwise. Moreover, some steps or constituent actions and/or a combination thereof in any given method or process may be skipped, omitted, spared and/or opted out, unless the context clearly dictates otherwise.

In the specification or claims herein, any term signifying an action or operation, such as: a verb, whether in base form or any tense, gerund or present/past participle, is not to be construed as necessarily to be actually performed but rather in a constructive manner, namely as to be performed merely optionally or potentially.

The term substantially as used herein is a broad term, and is to be given its ordinary and customary meaning to a person of ordinary skill in the art (and is not to be limited to a special or customized meaning), and refers without limitation to being largely but not necessarily entirely of that quantity or quality which is specified.

The term essentially means that the composition, method or structure may include additional ingredients, stages and or parts, but only if the additional ingredients, the stages and/or the parts do not materially alter the basic and new characteristics of the composition, method or structure claimed.

As used herein, the term essentially changes a specific meaning, meaning an interval of plus or minus ten percent (±10%). For any embodiments disclosed herein, any disclosure of a particular value, in some alternative embodiments, is to be understood as disclosing an interval approximately or about equal to that particular value (i.e., ±10%).

As used herein, the terms about or approximately modify a particular value, by referring to a range equal to the particular value, plus or minus twenty percent (+/−20%). For any of the embodiments, disclosed herein, any disclosure of a particular value, can, in various alternate embodiments, also be understood as a disclosure of a range equal to about that particular value (i.e. +/−20%).

As used herein, the term or is an inclusive or operator, equivalent to the term and/or, unless the context clearly dictates otherwise; whereas the term and as used herein is also the alternative operator equivalent to the term and/or, unless the context clearly dictates otherwise.

It should be understood, however, that neither the briefly synopsized summary nor particular definitions hereinabove are not to limit interpretation of the invention to the specific forms and examples but rather on the contrary are to cover all modifications, equivalents and alternatives falling within the scope of the invention.

### BRIEF DESCRIPTION OF THE DRAWINGS

The present invention will be understood and appreciated more comprehensively from the following detailed description taken in conjunction with the appended drawings in which:

FIG. 1 is a perspective view of an environmentally sustainable biogas production and buffered utilization system, according to some embodiments of the present invention;

FIG. 2 is an exploded perspective view of an environmentally sustainable biogas production and buffered utilization system, according to some embodiments of the present invention;

FIG. 3A is a perspective view of interior anaerobic digestor components, according to some embodiments of the present invention;

FIG. 3B is a perspective view of the components of the buffered utilization system, showing a feeder sub-assembly, a utilization module, an intermittent gas accumulation module and a sewage sub-system, according to some embodiments of the present invention;

FIG. 4A is a perspective view of the utilization module, according to some embodiments of the present invention;

FIG. 4B is a perspective view of the intermittent gas accumulation module, according to some embodiments of the present invention;

FIG. 4C is a perspective view of the feeder sub-assembly, according to some embodiments of the present invention;

FIG. 4D is a perspective view of the sewage sub-system of the system, according to some embodiments of the present invention;

FIG. 5 is a flowchart of a method of an environmentally sustainable method of biogas production and buffered utilization, according to some embodiments of the present invention.

While the invention is susceptible to various modifications and alternative forms, specific embodiments thereof have been shown merely by way of example in the drawings. The drawings are not necessarily complete and components are not essentially to scale; emphasis instead being placed upon clearly illustrating the principles underlying the present invention.

### DETAILED DESCRIPTION OF THE INVENTION

Illustrative embodiments of the invention are described below. In the interest of clarity, not all features of actual implementation are described in this specification. It should be appreciated that various features or elements described in the context of some embodiment may be interchangeable with features or elements of any other embodiment described in the specification. Moreover, it will be appreciated that for the development of any actual embodiment, numerous implementation-specific decisions must be made to achieve the developers' specific goals, such as compliance with technology-or business-related constraints, which may vary from one implementation to another, and the effort of such a development might be complex and time-consuming, but would nevertheless be a routine undertaking for those of ordinary skill in the art having the benefit of this disclosure.

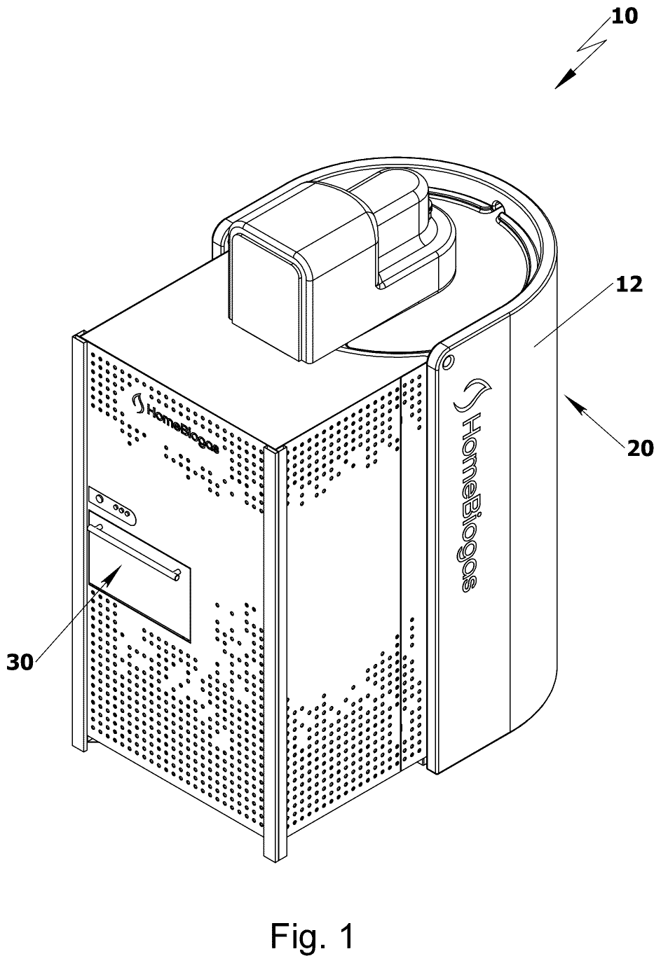

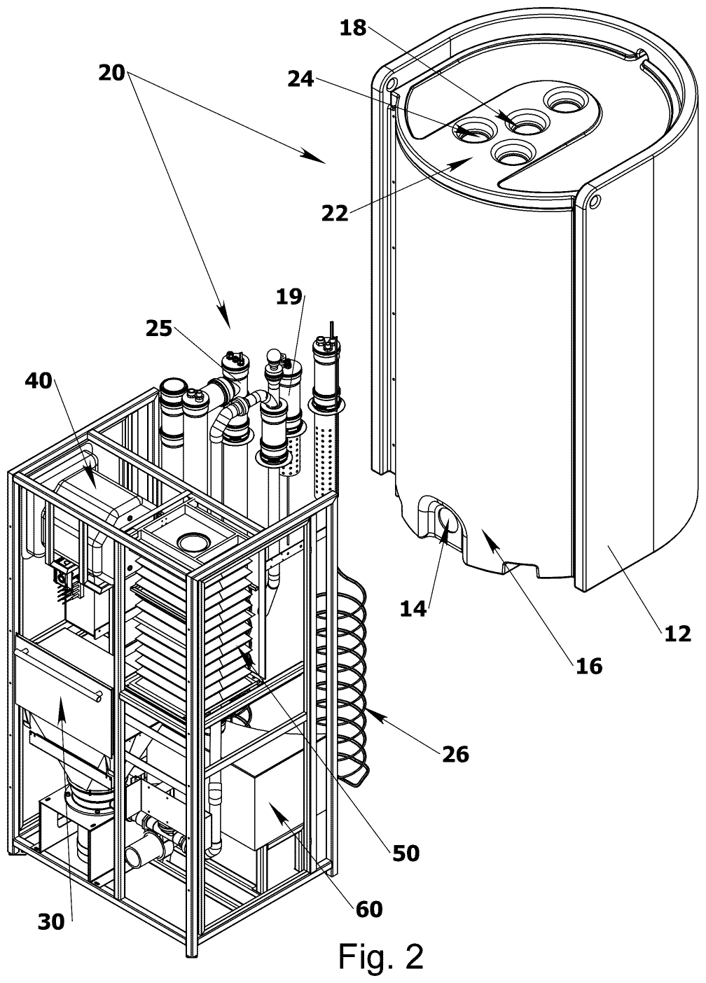

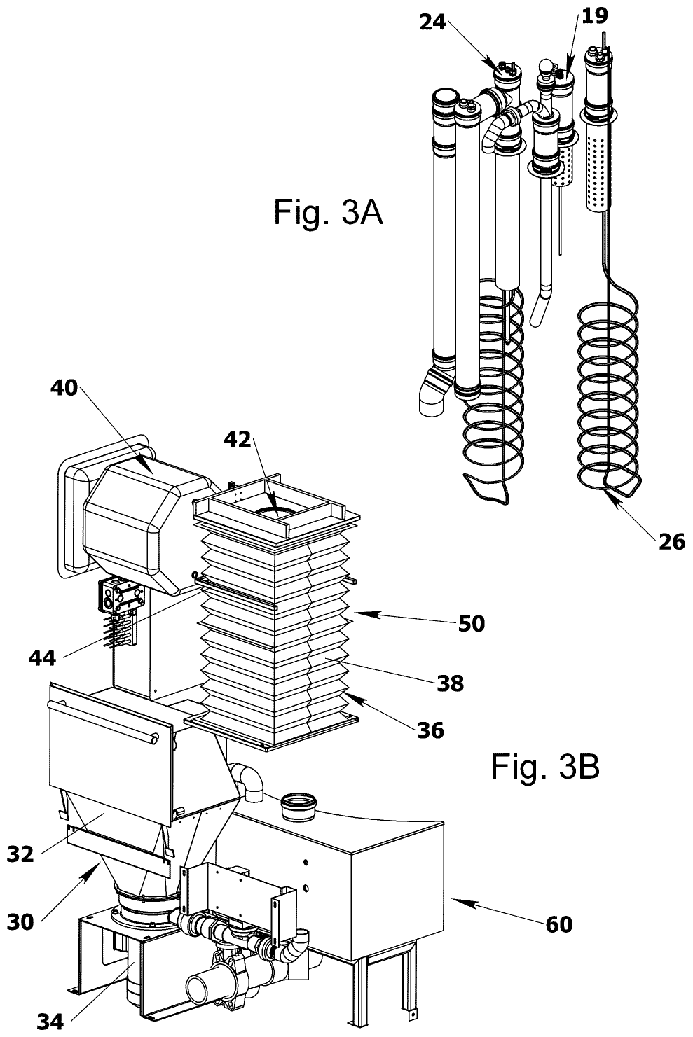

In accordance with some embodiments of the present invention, reference is now made to FIGS. 1 to 4D, showing environmentally sustainable biogas production and buffered utilization system 10. In some embodiments, system 10 comprises anaerobic digestor 20, feeder sub-assembly 30, utilization module 40 and intermittent gas accumulation module 50.

In some embodiments, anaerobic digestor 20 comprises essentially cylindrically shaped firm encasement 12. In some embodiments, cylindrically shaped firm encasement 12 further comprises an interior bladder, configured to facilitate the sealing of anaerobic digestor 20. In some embodiments, anaerobic digestor 20 further comprises inlet 14. Inlet 14 is typically disposed at bottom portion 16 of encasement 12. Inlet 14 is configured to receive ground organic waste from a grinder of feeder sub-assembly 30.

In some embodiments, anaerobic digestor 20 further comprises gas outlet 18. In some embodiments, anaerobic digestor 20 further comprises gas outlet pipe 19, hermetically attached to upper face 22 of encasement 12 of anaerobic digestor 20 and extending upwardly therefrom. Gas outlet 18 is configured to duct a biogas, produced by essentially anaerobic digest processes in anaerobic digestor 20 into gas outlet pipe 19.

In some embodiments, anaerobic digestor 20 further comprises overflow outlet 24 of encasement 12. In some embodiments, anaerobic digestor 20 further comprises overflow outlet pipe 25 hermetically attached to encasement 12 of anaerobic digestor 20 and extending downwardly therefrom.

In some embodiments, the inlet of overflow outlet pipe 25 is disposed at a centrical portion along the vertical length or height of encasement 12. Overflow outlet 24 is configured to drain surplus liquids from anaerobic digestor 20 via overflow outlet pipe 25. Positioning the inlet of overflow outlet pipe 25 at a centrical portion along the vertical length or height of encasement 12 is configured to avoid capturing solids whilst draining the surplus liquids from anaerobic digestor 20.

In some embodiments, anaerobic digestor 20 further comprises interior heater 26. Interior heater 26 is configured for interiorly heating anaerobic digestor 20. In some examples, interior heater 26 is an air heater module, a water heater module. In some embodiments, anaerobic digestor 20 is configured to be essentially filled with liquids, without a substantial space for a discrete gaseous fraction in encasement 12.

In some embodiments, system 10 comprises feeder sub-assembly 30. Feeder sub-assembly 30 is operationally connected to inlet 14. In some embodiments, feeder sub-assembly 30 comprises sink 32. Sink 32 is configured to receive organic waste and conveniently feeding-in organic waste into a grinder.

In some embodiments, feeder sub-assembly 30 further comprises grinder 34. Grinder 34 is operationally connected to sink 32 and to inlet 14. Grinder 34 is configured to receive the organic waste from sink 32, to grind the organic waste and to feed ground organic waste into inlet 14. In some embodiments, feeder sub-assembly 30 further comprises a buffer tank, disposed in-between grinder 34 and inlet 14.

In some embodiments, system 10 comprises utilization module 40. Utilization module 40 is operationally connected to gas outlet 18. Utilization module 40 is configured to receive the biogas from gas outlet 18 and to burn it upon demand.

In some embodiments, utilization module 40 comprises a controllable igniter. In some embodiments, utilization module 40 further comprises a gas consumer element, actuatable by the controllable igniter. The gas consumer element is configured for using up the biogas produced by anaerobic digester 20. In some examples, the at least one gas consumer of utilization module 40 is a gas burner, gas water heater, gas turbine and gas-powered electrical generator.

In some embodiments, utilization module 40 further comprises a controllable valve. The valve is configured to control an inflow of the biogas to the gas consumer element.

In some embodiments, system 10 comprises intermittent gas accumulation module 50. Intermittent gas accumulation module 50 comprises variable volume gas reservoir 36. Variable volume gas reservoir 36, made of a pliant material having a relatively high tensile strength, comprises elongated accordion shaped container 38.

In some embodiments, variable volume gas reservoir 36 is configured for forming a substantially constant positive pressure therein. In some embodiments, variable volume gas reservoir 36 is made up of a composite material, comprising a pliant polymeric sheet and a gas barrier film, preferably a metallic gas barrier film.

In some embodiments, variable volume gas reservoir 36 is configured to assume an erected configuration and a collapsed configuration. In some embodiments, in the erected configuration, variable volume gas reservoir 36 is essentially filled with the biogas. In some embodiments, in the collapsed configuration, variable volume gas reservoir 36 is essentially depleted of the biogas.

In some embodiments, intermittent gas accumulation module 50 further comprises at least one gas channel. At least one gas channel is operationally connected to gas outlet 18 and to utilization module 40.

In some embodiments, intermittent gas accumulation module 50 further comprises weight 42. Weight 42 is disposed on top portion 43 of variable volume gas reservoir 36. Weight 42 is configured to exert a predetermined gravitational force onto variable volume gas reservoir 36, thereby forming a substantially constant pressure, whilst variable volume gas reservoir 36 is in-between the erected and collapsed configurations.

In some embodiments, intermittent gas accumulation module 50 further comprises at least one sensor. In some embodiments, at least one sensor is disposed on top of weight 42. The at least one sensor is configured to detect variable volume gas reservoir 36 in the erected configuration. The sensor is activated when the sensed target is in a certain distance. The distance can be approximately less than 1 mm or in the range of about 1 mm to about 50 mm. In some embodiments, the sensor is connected to the sensing target. In some embodiments, the sensor is external to the sensing target. In some embodiments, the sensor is only activated upon direct contact (for example, maximum sensing distance is greater than zero).

In some examples, the at least one sensor is at least one of various means to determine presence or absence at a particular location or height, in a non-limiting manner including: optical sensors, optical reflecting sensors, LED/photodiode pair optical sensors, LED/phototransistor pair optical sensors, laser diode/photodiode pair optical sensors, laser diode/phototransistor pair optical sensors, optocouplers, optical fiber coupled optical sensors, magnetic sensors, weight sensors, force sensors, displacement sensors, pressure sensors, inductive sensors, magnetic sensors, capacitive sensors, microswitch, mechanical sensors, electro mechanical sensors, capacitive touch device, electric resistance sensor, inductance sensor, eddy current sensor, photoelectric sensor, supersonic sensing device, hall effect sensor, infrared sensors, touch sensors or surface acoustic wave (SAW) sensor, encoder, drop wire sensor, volume measuring sensor and/or any combination thereof.

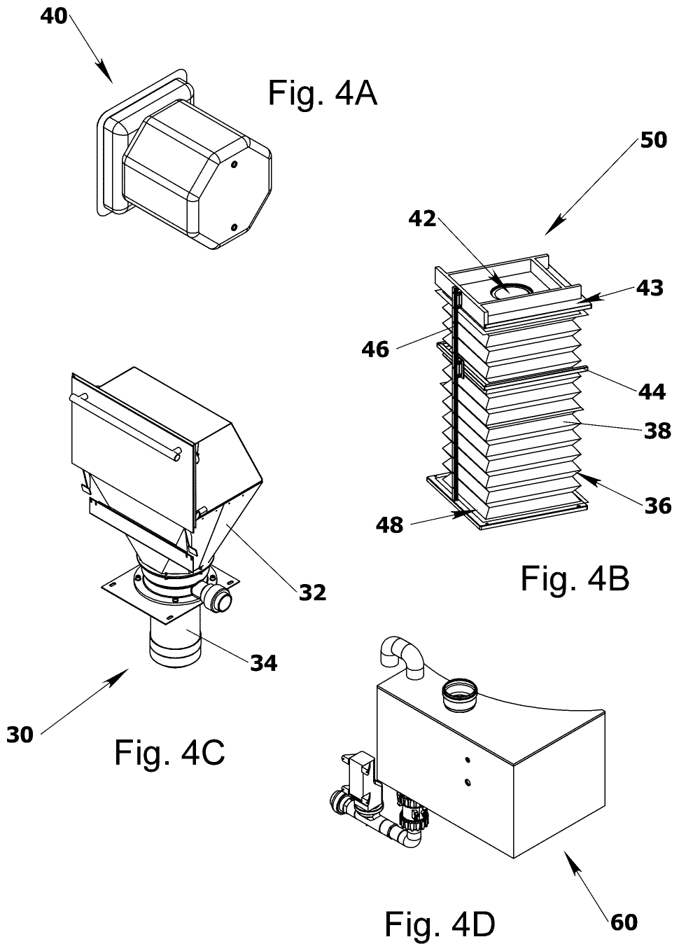

In some embodiments, intermittent gas accumulation module 50 further comprises mechanical restrictor 44. Mechanical restrictor 44 is configured to confine variable volume gas reservoir 36, thereby essentially preventing buckling and/or sidewise deformation of variable volume gas reservoir 36, from a substantially linearly straight vertically oriented confirmation, as shown in FIG. 4B.

In some embodiments, mechanical restrictor 44 comprises vertically slidable bracket 46. Vertically slidable bracket 46 is preferably movable together with variable volume gas reservoir 36, whilst variable volume gas reservoir 36 is transitional in-between the erected and collapsed configurations.

In some embodiments, mechanical restrictor 44 is disposed at a distance comprising approximately of ⅔ of a maximal length or height of variable volume gas reservoir 36, from bottom portion 48 of variable volume gas reservoir 36, whilst variable volume gas reservoir 36 is in the erected configuration, as shown in FIG. 4B.

In some embodiments, system 10 comprises a controller. The controller is operationally connected at least to the sensor of intermittent gas accumulation module 50 and the controllable igniter of utilization module 40. The controller is typically further operationally connected to the valve of utilization module 40.

In some embodiments, system 10 further comprises sewage sub-system 60. Sewage sub-system 60 is configured to receive at least one substance and then to feed it into inlet 14 of anaerobic digestor 20. In some examples, the at least one substance is greywater, blackwater and wastewater. In some embodiments, sewage sub-system 60 comprises a storage tank, configured to accumulate a sufficient amount of the substance prior to feeding it into inlet 14 of anaerobic digestor 20. In some embodiments, sewage sub-system 60 comprises a storage tank, configured to store an excessive amount of the substance prior to gradually feeding it into inlet 14 of anaerobic digestor 20.

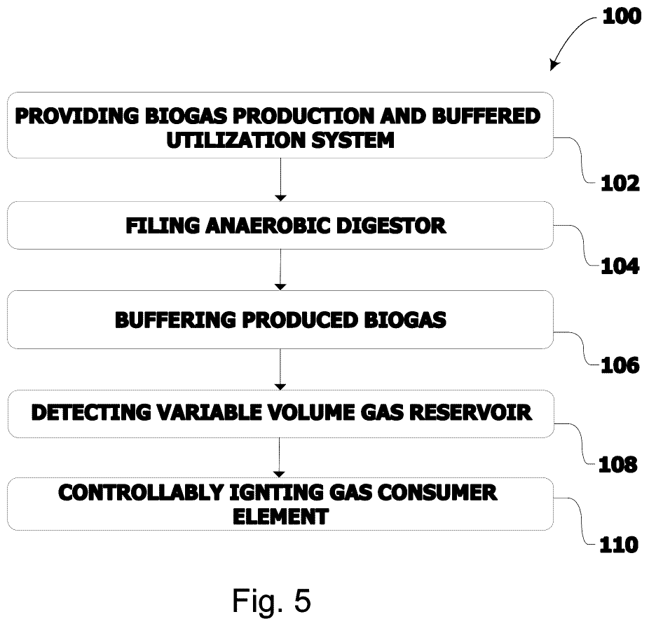

In accordance with some embodiments of the present invention, reference is now made FIG. 5 showing a flowchart of method 100 of an environmentally sustainable method of biogas production and buffered utilization. Method 100 of the embodiment of FIG. 5 illustrates various features that may be interchangeable with elements of any other embodiment described in the specification.

In some embodiments, method 100 commences at step 102 of providing a biogas production and buffered utilization system. In some embodiments, the biogas production and buffered utilization system comprises an anaerobic digestor. In some embodiments, the anaerobic digestor comprises an essentially cylindrically shaped firm encasement, an inlet disposed at a bottom portion of the encasement. In some embodiments, the anaerobic digestor further comprises a gas outlet disposed at a top portion of the encasement, configured to duct a biogas, produced by essentially anaerobic digest processes in the digestor. In some embodiments, the anaerobic digestor further comprises an overflow outlet of the encasement, configured to drain surplus liquids from the digestor.

In some embodiments, the biogas production and buffered utilization system further comprises a feeder sub-assembly operationally connected to the inlet. In some embodiments, the biogas production and buffered utilization system further comprises a utilization module operationally connected to the gas outlet, configured to receive the biogas from the gas outlet and to burn it upon demand.

In some embodiments, the biogas production and buffered utilization system further comprises an intermittent gas accumulation module. In some embodiments, the intermittent gas accumulation module comprises a variable volume gas reservoir, comprising an elongated pliant accordion shaped container. In some embodiments, the variable volume gas reservoir is configured to assume an erected configuration, in which the variable volume gas reservoir is essentially filled with the biogas and a collapsed configuration, in which the variable volume gas reservoir is essentially depleted of the biogas.

In some embodiments, the intermittent gas accumulation module further comprises at least one gas channel, operationally connected to the gas outlet and to the utilization module. In some embodiments, the intermittent gas accumulation module further comprises a weight disposed on top of the variable volume gas reservoir, configured to exert a predetermined gravitational force onto the variable volume gas reservoir, thereby forming a substantially constant pressure, whilst the variable volume gas reservoir is in-between the erected and collapsed configurations. In some embodiments, the intermittent gas accumulation module further comprises at least one sensor, disposed on top of the weight, configured to detect the variable volume gas reservoir in the erected configuration.

In some embodiments, the biogas production and buffered utilization system further comprises a controller, operationally connected at least to the sensor of the intermittent gas accumulation module and the controllable igniter of the utilization module.

In some embodiments, method 100 proceeds to step 104 of filling the anaerobic digestor with liquids, without leaving a substantial discrete space for a gaseous fraction in the encasement.

In some embodiments, method 100 further proceeds to step 106 of buffering a produced biogas in the intermittent gas accumulation module, by changing a volume of the variable volume gas reservoir.

In some embodiments, method 100 yet further proceeds to step 108 of detecting by the sensor that the variable volume gas reservoir in the erected configuration. In some embodiments, method 100 comprises step 110 of controllably igniting the gas consumer element by the igniter. In some embodiments, step 110 of method 100 includes conditionally igniting the gas consumer element by the igniter, only upon detecting by the sensor that the variable volume gas reservoir in the erected configuration at step 108.

In some embodiments, step 110 of method 100 comprises opening the valve of the utilization module and forming the inflow of the biogas to the gas consumer element. In some embodiments, step 110 of method 100 further comprises closing the valve of the utilization module and obstructing the inflow of said biogas to the gas consumer element.

In some embodiments, the intermittent gas accumulation module further comprises a mechanical restrictor, configured to confine the variable volume gas reservoir. In some embodiments, method 100 further comprises preventing buckling and/or sidewise deformation of the variable volume gas reservoir, from a substantially linearly straight vertically oriented conformation, by the mechanical restrictor.

In some embodiments, the mechanical restrictor comprises a vertically slidable bracket. In some embodiments, method 100 further comprises moving the vertically slidable bracket with the variable volume gas reservoir, whilst the variable volume gas reservoir is in-between the erected and collapsed configurations.

In some embodiments, method 100 further comprises disposing the mechanical restrictor at a distance comprising of approximately ⅔ of a maximal length or height of the variable volume gas reservoir, from a bottom of the variable volume gas reservoir, whilst the variable volume gas reservoir is in the erected configuration.

In some embodiments, method 100 further comprises positioning the overflow outlet at a centrical portion along a length of the encasement and avoiding capturing solids whilst draining the surplus liquids from the digestor.

In some embodiments, method 100 further comprises interiorly heating said anaerobic digestor.

It will be appreciated by persons skilled in the art of the invention that various features and/or elements elaborated in the context of a specific embodiment described hereinabove and/or referenced herein and/or illustrated by a particular example in a certain drawing enclosed hereto, whether method, system, device or product, is/are interchangeable with features and/or elements of any other embodiment described in the specification and/or shown in the drawings. Moreover, skilled persons would appreciate that the present invention is not limited by what has been particularly shown and described hereinabove. Rather the scope of the invention is defined by the claims which follow:

1. An environmentally sustainable biogas production and buffered utilization system comprises:
a. an anaerobic digestor comprising:
(I) an essentially cylindrically shaped firm encasement;
(II) an inlet configured to sustain a feed into said encasement;
(III) a gas outlet disposed at a top portion of said encasement, configured to duct a biogas, produced by essentially anaerobic digest processes in said digestor;
(IV) an overflow outlet of said encasement, configured to drain surplus liquids from said digestor;
wherein said anaerobic digestor is configured to be essentially filled with liquids, without a substantial space for a discrete gaseous fraction in said encasement;
b. a feeder sub-assembly operationally connected to said inlet, comprising:
(I) a sink configured to receive organic waste;
(II) a grinder, operationally connected to said sink and to said inlet, configured to receive said organic waste from sink, to grind said organic waste and to feed ground organic waste into said inlet;
c. a utilization module operationally connected to said gas outlet, configured to receive said biogas from said gas outlet and to burn it, comprising:
(I) a controllable igniter;
(II) a gas consumer element, actuatable by a controller;
(III) a valve configured to control an inflow of said biogas to said gas consumer element;
d. an intermittent gas accumulation module comprising:
(I) a variable volume gas reservoir, comprising an elongated pliant accordion shaped container, said variable volume gas reservoir is configured to assume at least:
(i) an erected configuration, wherein said variable volume gas reservoir is essentially filled with said biogas, and
(ii) a collapsed configuration, wherein said variable volume gas reservoir is essentially depleted of said biogas;
(II) at least one gas channel, operationally connected to said gas outlet and to said utilization module;
(III) a weight disposed on top of said variable volume gas reservoir, configured to exert a predetermined gravitational force onto said variable volume gas reservoir, thereby forming a substantially constant pressure, whilst said variable volume gas reservoir is in-between said erected and collapsed configurations;
(IV) at least one sensor, configured to detect said variable volume gas reservoir in said erected configuration;
e. a controller, operationally connected at least to said sensor of said intermittent gas accumulation module and said controllable igniter of said utilization module.

2. The system as, in claim 1, further comprises a sewage sub-system, configured to receive at least one substance selected from the group consisting of: greywater, blackwater and wastewater.

3. The system as in claim 1, wherein said anaerobic digestor further comprises an interior heater.

4. The system as in claim 1, wherein said utilization module comprises at least one gas consumer device selected from the group consisting of: a gas burner, gas water heater, gas turbine and gas powered electrical generator.

5. The system as in claim 1, wherein said variable volume gas reservoir comprises a composite material, comprising a pliant polymeric sheet and a gas barrier film.

6. The system as in claim 1, wherein said controller is further operationally connected to said valve of said utilization module.

7. The system as in claim 1, wherein said intermittent gas accumulation module further comprises a mechanical restrictor, configured to confine said variable volume gas reservoir, thereby essentially preventing buckling and/or sidewise deformation of said variable volume gas reservoir, from a substantially linearly straight vertically oriented conformation.

8. The system as in claim 7, wherein said mechanical restrictor comprises a vertically slidable bracket, movable with said variable volume gas reservoir, whilst said variable volume gas reservoir is in-between said erected and collapsed configurations.

9. The system as in claim 7, wherein said mechanical restrictor comprises a plurality vertically slidable brackets, disposed equidistantly alongside said variable volume gas reservoir is in said erected configuration.

10. The system as in claim 1, wherein said overflow outlet is disposed at a centrical portion along a length of said encasement, configured to avoid capturing solids whilst draining said surplus liquids from said digestor.

11. An environmentally sustainable method of biogas production and buffered utilization comprises:
a. providing a biogas production and buffered utilization system comprising:
(I) an anaerobic digestor comprising:
(i) an essentially cylindrically shaped firm encasement;
(ii) an inlet configured to sustain a feed into said encasement;
(iii) a gas outlet disposed at a top portion of said encasement, configured to duct a biogas, produced by essentially anaerobic digest processes in said digestor;
(iv) an overflow outlet of said encasement, configured to drain surplus liquids from said digestor;
(II) a feeder sub-assembly operationally connected to said inlet, comprising:
(i) a sink configured to receive organic waste;
(ii) a grinder, operationally connected to said sink and to said inlet, configured to receive said organic waste from sink, to grind said organic waste and to feed ground organic waste into said inlet;
(III) a utilization module operationally connected to said gas outlet, configured to receive said biogas from said gas outlet and to burn it, comprising:
(i) a controllable igniter;
(ii) a gas consumer element, actuatable by a controller;
(iii) a valve configured to control an inflow of said biogas to said gas consumer element;
(IV) an intermittent gas accumulation module;
(i) a variable volume gas reservoir, comprising an elongated pliant accordion shaped container, said variable volume gas reservoir is configured to assume at least: an erected configuration, wherein said variable volume gas reservoir is essentially filled with said biogas and a collapsed configuration, wherein said variable volume gas reservoir is essentially depleted of said biogas;
(ii) at least one gas channel, operationally connected to said gas outlet and to said utilization module;
(iii) a weight disposed on top of said variable volume gas reservoir, configured to exert a predetermined gravitational force onto said variable volume gas reservoir, thereby forming a substantially constant pressure, whilst said variable volume gas reservoir is in-between said erected and collapsed configurations;
(iv) at least one sensor, configured to detect said variable volume gas reservoir in said erected configuration;
(V) a controller, operationally connected at least to said sensor of said intermittent gas accumulation module and said controllable igniter of said utilization module;
b. filling said anaerobic digestor with liquids, without leaving a substantial discrete space for a gaseous fraction in said encasement;
c. buffering a produced biogas in said intermittent gas accumulation module, by changing a volume of said variable volume gas reservoir;
d. detecting by said sensor said variable volume gas reservoir in said erected configuration;
e. controllably igniting said gas consumer element by said igniter.

12. The method as in claim 11, wherein said system further comprising a sewage sub-system, further comprises receiving at least one substance selected from the group consisting of: greywater, blackwater and wastewater.

13. The method, as in claim 11, further comprises interiorly heating said anaerobic digestor.

14. The method as in claim 11, wherein said utilization module comprises at least one gas consumer device selected from the group consisting of: a gas burner, gas water heater, gas turbine and gas powered electrical generator.

15. The method as in claim 11, wherein said variable volume gas reservoir comprises a composite material, comprising a pliant polymeric sheet and a gas barrier film.

16. The method as in claim 11, wherein said controller is further operationally connected to said valve of said utilization module, further comprises:
a. opening said valve of said utilization module and forming said inflow of said biogas to said gas consumer element, and
b. closing said valve of said utilization module and obstructing said inflow of said biogas to said gas consumer element.

17. The method as in claim 11, wherein said intermittent gas accumulation module further comprises a mechanical restrictor, configured to confine said variable volume gas reservoir, further comprises preventing buckling and/or sidewise deformation of said variable volume gas reservoir, from a substantially linearly straight vertically oriented conformation, by said mechanical restrictor.

18. The method as in claim 17, wherein said mechanical restrictor comprises a vertically slidable bracket, further comprises moving said vertically slidable bracket with said variable volume gas reservoir, whilst said variable volume gas reservoir is in-between said erected and collapsed configurations.

19. The method, as in claim 17, wherein said mechanical restrictor comprises a plurality vertically slidable brackets, disposed equidistantly alongside said variable volume gas reservoir is in said erected configuration.

20. The method, as in claim 11, further comprises positioning said overflow outlet at a centrical portion along a length of said encasement and avoiding capturing solids whilst draining said surplus liquids from said digestor.
